(19) United States
(12) Patent Application Publication
(10) Pub. No.: US 2005/0019358 A1
(43) Pub. Date: Jan. 27, 2005

(54) Method for providing stability to cosmetic compositions containing heteropolymers and oil-soluble esters and methods of using same

(75) Inventors: Carlos Pinzon, New Milford, NJ (US); Paul Thau, Berkeley heights, NJ (US)

Correspondence Address: FINNEGAN, HENDERSON, FARABOW, GARRETT & DUNNER;LLP, 1300 I STREET, NW, WASHINGTON, DC, 20005, US

(73) Assignee: L'Oreal S.A.

(21) Appl. No.: 10/918,579

(22) Filed: Aug. 16, 2004

Related U.S. Application Data
(63) Continuation of application No. 09/733,898, filed on Dec. 12, 2000.

(51) Int. Cl. A61K 7/00

(52) U.S. Cl. 424/401

(57) ABSTRACT

A method for providing stability to cosmetic compositions comprising at least one structuring polymer, wherein said at least one structuring polymer is chosen from polyamide polymers of formula (I): 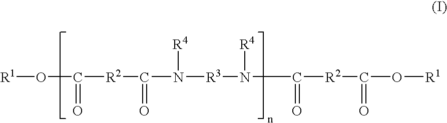 in which:
n is an integer which represents the number of amide units such that the number of ester groups present in said at least one polyamide polymer ranges from 10% to 50% of the total number of all said ester groups and all said amide groups comprised in said at least one polyamide polymer; R¹, which are identical or different, are each chosen from alkyl groups having at least 4 carbon atoms and alkenyl groups having at least 4 carbon atoms; R², which are identical or different, are each chosen from C₄ to C₄₂ hydrocarbon-based groups with the proviso that at least 50% of all R² are chosen from C₃₀ to C₄₂ hydrocarbon-based groups; R³, which are identical or different, are each chosen from C₂ to C₃₆ hydrocarbon-based groups; and R⁴, which are identical or different, are each chosen from hydrogen and C₁ to C₁₀ alkyl groups, with the proviso that at least 50% of all R⁴ are chosen from hydrogen; and at least one at least one oil-soluble ester comprising at least one free hydroxy group. The compositions may further comprise at least one fatty alcohol. This invention may be in the form of stable compositions such as, for example, mascara, make-up sticks, lipsticks and sunscreen sticks.

The present invention relates to compositions and methods for care of, for treating, and for making-up at least one keratinous material, in particular at least one human keratinous material, such as skin, including the scalp, and the lips, and/or at least one keratinous fiber which includes hair, eyelashes, and eyebrows. More particularly, the compositions of the invention comprise at least one structuring polymer and at least one oil-soluble ester comprising at least one free hydroxy group with the proviso that said at least one oil-soluble ester is not castor oil. The compositions may further comprise at least one fatty alcohol. This invention may be in the form of a stable composition such as, for example, make-up sticks, lipsticks, transparent sticks' and sunscreen sticks. The compositions may also, for example, result in a molded composition.

The use of high molecular weight polyamides to produce clear stick compositions dates back to the mid 1960's. These systems contained a combination of polyamide polymer, castor oil, esters, amides, and colorants as described in, for example, U.S. Pat. Nos. 3,086,914 and 3,148,125. However, there were significant drawbacks associated with such compositions. For example, the sticks were tacky and difficult to apply to the lips. During storage, particularly at slightly elevated temperatures, the stick surface developed distinct oil droplets (syneresis) which were not reabsorbed after the stick cooled to normal room temperature.

There have been many attempts to resolve the aforementioned technical problems with only partial success. The introduction of new specialty cosmetic esters has made it possible to reduce product tackiness and thereby improve application characteristics. However, these modifications did not diminish the tendency of these formulations to have stability problems such as developing distinct and unattractive oil syneresis. In some instances, these modified formulations also displayed poor temperature stability at 50° C.

The inventors have found that the use of specific structuring polymers and oil-soluble esters comprising at least one free hydroxy group may result in a stable composition. In one embodiment, the compositions of the invention also may provide good gelling efficiency and/or maintain desirable cosmetic application properties.

In one embodiment, the invention provides a composition comprising at least one structuring polymer comprising a polymer skeleton which comprises at least one hydrocarbon-based repeating unit comprising at least one hetero atom. The composition further comprises at least at least one oil-soluble ester with the proviso that said at least one oil-soluble ester is not castor oil. In a further embodiment, the at least one structuring polymer and the at least one oil-soluble ester are present in a combined amount effective to stabilize the composition. As used herein, the expression “at least one” means one or more and thus includes individual components as well as mixtures and combinations thereof.

The invention also provides a method for providing stability to a composition comprising including in the composition at least one structuring polymer comprising a polymer skeleton which comprises at least one hydrocarbon-based repeating unit comprising at least one hetero atom. The composition further comprises at least at least one oil-soluble ester with the proviso that said at least one oil-soluble ester is not castor oil.

The invention also provides for a cosmetic process for caring for, making up, and/or treating a keratinous material comprising applying to at least one keratinous material a cosmetic composition comprising at least one structuring polymer comprising a polymer skeleton which comprises at least one hydrocarbon-based repeating unit comprising at least one hetero atom. The composition further comprises at least at least one oil-soluble ester with the proviso that said at least one oil-soluble ester is not castor oil. As used herein, “keratinous material” is meant to comprise hair, lips, skin, scalp and superficial body growths such as eyelashes, eyebrows and nails.

It is to be understood that both the foregoing general description and the following detailed description are exemplary and explanatory only and are not restrictive of the invention as claimed.

One subject of the invention is cosmetic and/or dermatological compositions which are useful for the care, make-up and/or treatment of at least one keratinous material which may be of suitable hardness to allow preparation of these compositions in the form of a stick or other structured form which may be stable.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as bending or leaning if the composition is in stick form, phase separation, melting, or syneresis. As used herein, syneresis is the appearance of droplets on the surface of a composition that are visible to the naked eye. Syneresis or oil release from a composition, such as a stick, that is only apparent as a thin, attractive, and glossy, surface coating is not considered a composition that has failed the stability test. The stability is further tested by repeating the 8 week test at 4° C., 37° C., 45° C., and 50° C., and under freeze-thaw conditions. A composition is considered to lack stability if an abnormality that impedes functioning of the composition is observed in any of these tests. The skilled artisan will readily recognize an abnormality that impedes functioning of a composition based on the intended application.

The invention applies not only to make-up products for at least one keratinous material such as lip compositions, lip pencils, foundations including foundations which may be cast in the form of a stick or a dish, concealer products, temporary tattoo products, eyeliners, and mascara bars, but also to body hygiene products such as deodorant sticks, and to care products and products, for treating at least one keratinous material such as sunscreen (anti-sun) and after-sun products which may be in stick form and also nail products. It is to be noted that a deodorant product is a body hygiene product and does not relate to care, make-up, or treatment of keratin materials, including keratinous fibers, skin, or lips.

The present invention may be in the form of a mascara product, an eyeliner product, a foundation product, a lipstick product, a lip balm, a blush for cheeks or eyelids, a deodorant product, a fragrance product, a make-up product for the body, a make-up-removing product, an eyeshadow product, a face powder product, a night or day care product for the face, a concealer product, a hair conditioning product, a sun screen, a colorant for the skin or hair, or a skin care formula such as, for example, anti-pimple, or shaving cut formulas. According to one embodiment of the invention, the composition is in the form of a substantially clear or substantially transparent composition such as, for example, a clear lipstick, clear sunscreen composition, or clear foundation, in particular for concealing skin imperfections.

For example, the composition of the present invention may be in a form chosen from a paste, a solid, a gel, and a cream. It may be an emulsion, i.e., an oil-in-water or water-in-oil emulsion, a multiple emulsion, e.g., an oil-in-water-in-oil emulsion or water-in-oil-in-water emulsion, or a solid, rigid, or supple gel, including anhydrous gels. In one embodiment, the composition of the invention comprises an external fatty phase or a continuous fatty phase, which fatty phase can be anhydrous. In another embodiment, the composition of the invention is transparent or clear. The composition can also be in a form chosen from a translucent anhydrous gel and a transparent anhydrous gel. The composition can also be a molded composition or cast as a stick or a dish. The composition in one embodiment is a solid or rigid product, such as a molded stick or a poured stick.

Structuring Polymer

In one embodiment, the at least one structuring polymer in the composition of the invention is a solid that is not deformable at room temperature (25° C.) and atmospheric pressure (760 mmHg). In a further embodiment, the at least one structuring polymer is capable of structuring the composition without opacifying it. As defined above, the at least one structuring polymer of the present invention comprises a polymer skeleton comprising at least one hydrocarbon-based repeating unit comprising at least one hetero atom. In one embodiment, the at least one structuring polymer further comprises at least one terminal fatty chain chosen from alkyl and alkenyl chains, such as of at least 4 atoms, and further such as comprising 8 to 120 carbon atoms, bonded to the polymer skeleton via at least one linking group. The terminal fatty chain may, for example, be functionalized. The at least one structuring polymer may also further comprise at least one pendant fatty chain chosen from alkyl and alkenyl chains, such as of at least 4 atoms, and further such as comprising 8 to 120 carbon atoms, bonded to any carbon or hetero atom of the polymer skeleton via at least one linking group. The pendant fatty chain may, for example, be functionalized. The at least one structuring polymer may comprise both at least one pendant fatty chain and at least one terminal fatty chain as defined above, and one or both types of chains can be functionalized.

In one embodiment, the structuring polymer comprises at least two hydrocarbon-based repeating units. As a further example, the structuring polymer comprises at least three hydrocarbon-based repeating units and as an even further example, the at least three repeating units are identical.

As used herein, “functionalized” means comprising at least one functional group. Non-limiting examples of functional groups include hydroxyl groups, ether groups, oxyalkylene groups, polyoxyalkylene groups, carboxylic acid groups, amine groups, amide groups, halogen containing groups, including fluoro and perfluoro groups, halogen atoms, ester groups, siloxane groups and polysiloxane groups.

For purposes of the invention, the expression “functionalized chain” means, for example, an alkyl chain comprising at least one functional (reactive) group chosen, for example, from those recited above. For example, in one embodiment, the hydrogen atoms of at least one alkyl chain may be substituted at least partially with fluorine atoms.

According to the invention, these chains may be linked directly to the polymer skeleton or via an ester function or a perfluoro group.

For the purposes of the invention, the term “polymer” means a compound containing at least 2 repeating units, such as, for example, a compound containing at least 3 repeating units, which may be identical.

As used herein, the expression “hydrocarbon-based repeating unit” includes a repeating unit comprising from 2 to 80 carbon atoms, such as, for example, from 2 to 60 carbon atoms. The at least one hydrocarbon-based repeating unit may also comprise oxygen atoms. The hydrocarbon-based repeating unit may be chosen from saturated and unsaturated hydrocarbon-based repeating units which in turn may be chosen from linear hydrocarbon-based repeating units, branched hydrocarbon-based repeating units and cyclic hydrocarbon-based repeating units. The at least one hydrocarbon-based repeating unit may comprise, for example, at least one hetero atom that is part of the polymer skeleton, i.e., not pendant. The at least one hetero atom may be chosen, for example, from nitrogen, sulphur, and phosphorus. For example, the at least one hetero atom may be a nitrogen atom, such as a non-pendant nitrogen atom. In another embodiment, the at least one hydrocarbon-based repeating unit may comprise at least one hetero atom with the proviso that the at least one hetero atom is not nitrogen. In another embodiment, the at least one hetero atom is combined with at least one atom chosen from oxygen and carbon to form a hetero atom group. In one embodiment, the hetero atom group comprises a carbonyl group.

The at least one repeating unit comprising at least one hetero atom may be chosen, for example, from amide groups, carbamate groups, and urea groups. In one embodiment, the at least one repeating unit comprises amide groups forming a polyamide skeleton. In another embodiment, the at least one repeating unit comprises carbamate groups and/or urea groups forming a polyurethane skeleton, a polyurea skeleton and/or a polyurethane-polyurea skeleton. The pendant chains, for example, can be linked directly to at least one of the hetero atoms of the polymer skeleton. In yet another embodiment, the at least one hydrocarbon-based repeating unit may comprise at least one hetero atom group with the proviso that the at least one hetero atom group is not an amide group. In one embodiment, the polymer skeleton comprises at least one repeating unit chosen from silicone units and oxyalkylene units, the at least one repeating unit being between the hydrocarbon-based repeating units.

In one embodiment, the compositions of the invention comprise at least one structuring polymer with nitrogen atoms, such as amide, urea, or carbamate units, such as amide units, and at least one polar oil.

In one embodiment, in the at least one structuring polymer, the percentage of the total number of fatty chains ranges from 40% to 98% relative to the total number of repeating units and fatty chains, and as a further example, from 50% to 95%. In a further embodiment wherein the polymer skeleton is a polyamide skeleton, in the at least one structuring polymer, the percentage of the total number of fatty chains ranges from 40% to 98% relative to the total number of all amide units and fatty chains, and as a further example, from 50% to 95%.

In a further embodiment, the nature and proportion of the at least one hydrocarbon-based repeating unit comprising at least one hetero atom depends on the nature of a liquid fatty phase of the composition and is, for example, similar to the nature of the fatty phase. For example, not to be limited as to theory, the more polar the hydrocarbon-based repeating units containing a hetero atom, and in high proportion, which corresponds to the presence of several hetero atoms, the greater the affinity of the at least one structuring polymer to polar oils. Conversely, the more non-polar, or even apolar, and lesser in proportion the hydrocarbon-based repeating units containing a hetero atom, the greater the affinity of the polymer for apolar oils.

In another embodiment, the invention is drawn to a structured composition containing at least one liquid fatty phase structured with at least one structuring polymer, wherein said at least one structuring polymer is a polyamide comprising a polymer skeleton comprising at least one amide repeating unit and optionally at least one pendant fatty chain and/or at least one terminal chain that are optionally functionalized and comprise from 8 to 120 carbon atoms, bonded to at least one of the amide repeating units via at least one linking group. The liquid fatty phase further contains at least one oil-soluble ester comprising at least one free hydroxy group with the proviso that said at least one oil-soluble ester is not castor oil. The at least one liquid fatty phase, the at least one structuring polyamide and the at least one oil-soluble ester comprising at least one free hydroxy group with the proviso that said at least one oil-soluble ester is not castor oil together form a physiologically acceptable medium.

When the structuring polymer has amide repeating units, the pendant fatty chains may be linked to at least one of the nitrogen atoms in the amide repeating units.

The structuring polymer, for example the polyamide polymer, may have a weight-average molecular mass of less than 100,000, such as less than 50,000. In another embodiment, the weight-average molecular mass may range from 1000 to 30,000, such as from 2000 to 20,000, further such as from 2000 to 10,000.

As discussed, the at least one structuring polymer may, for example, be chosen from polyamide polymers. A polyamide polymer may comprise, for example, a polymer skeleton which comprises at least one amide repeating unit, i.e., a polyamide skeleton. In one embodiment, the polyamide skeleton may further comprise at least one terminal fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group, and/or at least one pendant fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group. In one embodiment, the polyamide skeleton may comprise at least one terminal fatty chain chosen from fatty chains comprising 8 to 120 carbon atoms, such as, for example, 12 to 68 carbon atoms, bonded to the at least one polyamide skeleton via at least one linking group and/or at least one pendant fatty chain chosen chosen from fatty chains comprising 8 to 120 carbon atoms, such as, for example, 12 to 68 carbon atoms, bonded to the at least one polyamide skeleton via at least one linking group, such as bonded to any carbon or nitrogen of the polyamide skeleton via said at least one linking group. In one embodiment, the at least one linking group is chosen from single bonds and urea, urethane, thiourea, thiourethane, thioether, thioester, ester, ether and amine groups. For example, the at least one linking group may be chosen from ureas, esters and amines, and in another example, from esters and amines. The bond is, for example, an ester bond. In one embodiment, these polymers comprise a fatty chain at each end of the polymer skeleton, such as the polyamide skeleton.

In one embodiment, due to the presence of at least one chain, the polyamide polymers may be readily soluble in oils (i.e., water-immiscible liquid compounds) and thus may give macroscopically homogeneous compositions even with a high content (at least 25%) of the polyamide polymers, unlike certain polymers of the prior art that do not contain such alkyl or alkenyl chains at the end of the polyamide skeleton. As defined herein, a composition is soluble if it has a solubility of greater than 0.01 g per 100 ml of solution at 25° C.

In a further embodiment, the polyamide polymers can be chosen from polymers resulting from at least one polycondensation reaction between at least one acid chosen from dicarboxylic acids comprising at least 32 carbon atoms, such as 32 to 44 carbon atoms, and at least one amine chosen from diamines comprising at least 2 carbon atoms, such as from 2 to 36 carbon atoms, and triamines comprising at least 2 carbon atoms, such as from 2 to 36 carbon atoms. The dicarboxylic acids can, for example, be chosen from dimers of at least one fatty acid comprising at least 16 carbon atoms, such as oleic acid, linoleic acid and linolenic acid. The at least one amine can, for example, be chosen from diamines, such as ethylenediamine, hexylenediamine, hexamethylenediamine, phenylenediamine and triamines, such as ethylenetriamine.

The polyamide polymers may also be chosen from polymers comprising at least one terminal carboxylic acid group. The at least one terminal carboxylic acid group can, for example, be esterified with at least one alcohol chosen from monoalcohols comprising at least 4 carbon atoms. For example, the at least one alcohol can be chosen from monoalcohols comprising from 10 to 36 carbon atoms. In a further embodiment, the monoalcohols can comprise from 12 to 24 carbon atoms, such as from 16 to 24 carbon atoms, and for example 18 carbon atoms.

In one embodiment, the at least one polyamide polymer may be chosen from those described in U.S. Pat. No. 5,783,657, the disclosure of which is incorporated herein by reference, which are polyamide polymers of formula (I): 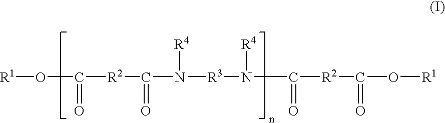
in which:
n is an integer which represents the number of amide units such that the number of ester groups present in said at least one polyamide polymer ranges from 10% to 50% of the total number of all said ester groups and all said amide groups comprised in the at least one polyamide polymer;
R¹, which are identical or different, are each chosen from alkyl groups comprising at least 4 carbon atoms and alkenyl groups comprising at least 4 carbon atoms. In one embodiment, the alkyl group comprises from 4 to 24 carbon atoms and the alkenyl group comprises from 4 to 24 carbon atoms;
R², which are identical or different, are each chosen from C₄ to C₄₂ hydrocarbon-based groups with the proviso that at least 50% of all R² are chosen from C₃₀ to C₄₂ hydrocarbon-based groups;
R³, which are identical or different, are each chosen from organic groups comprising atoms chosen from carbon atoms, hydrogen atoms, oxygen atoms and nitrogen atoms with the proviso that R³ comprises at least 2 carbon atoms; and
R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

In the polymer of formula (1), the terminal fatty chains that are optionally functionalized for the purposes of the invention are terminal chains linked to the last hetero atom, in this case nitrogen, of the polyamide skeleton.

In one embodiment, the ester groups of formula (I), which form part of the terminal and/or pendant fatty chains for the purposes of the invention, are present in an amount ranging from 15% to 40% of the total number of ester and amide groups, such as from 20% to 35%.

In formula (I), in one embodiment, n may be an integer ranging from 1 to 5, for example an integer ranging from 3 to 5. In the present invention, R¹, which are identical or different, can, for example, each be chosen from C₁₂ to C₂₂ alkyl groups, such as from C₁₆ to C₂₂ alkyl groups.

In the present invention, R², which are identical or different, can, for example, each be chosen from C₁₀ to C₄₂ hydrocarbon-based, e.g., alkylene groups. At least 50% of all R², for example at least 75% of all R², which are identical or different, can, for example, each be chosen from groups comprising from 30 to 42 carbon atoms. In the two aforementioned embodiments, the remaining R², which are identical or different, can, for example, each be chosen from C₄ to C₁₈ groups, such as C₄ to C₁₂ groups

R³, which can be identical or different, can, for example, each be chosen from C₂ to C₃₆ hydrocarbon-based groups and polyoxyalkylene groups. In another example, R³, which can be identical or different, can each, for example, be chosen from C₂ to C₁₂ hydrocarbon-based groups. In another embodiment, R⁴, which can be identical or different, can each be chosen from hydrogen atoms. As used herein, hydrocarbon-based groups may be chosen from linear, cyclic and branched, and saturated and unsaturated groups. The hydrocarbon-based groups can be chosen from aliphatic and aromatic groups. In one example, the hydrocarbon-based groups are chosen from aliphatic groups. The alkyl and alkylene groups may be chosen from linear, cyclic and branched, and saturated and unsaturated groups.

In general, the pendant and terminal fatty chains may be chosen from linear, cyclic and branched, and saturated and unsaturated groups. The pendant and terminal fatty chains can be chosen from aliphatic and aromatic groups. In one example, the pendant and terminal fatty chains are chosen from aliphatic groups.

According to the invention, the structuring of the liquid fatty phase is obtained with the aid of at least one structuring polymer, such as the at least one polymer of formula (I). The at least one polyamide polymer of formula (I) may, for example, be in the form of a mixture of polymers, and this mixture may also comprise a compound of formula (I) wherein n is equal to zero, i.e., a diester.

Non-limiting examples of an at least one polyamide polymer which may be used in the composition according to the present invention include the commercial products sold by Arizona Chemical under the names Uniclear 80 and Uniclear 100. These are sold, respectively, in the form of an 80% (in terms of active material) gel in a mineral oil and a 100% (in terms of active material) gel. These polymers have a softening point ranging from 88° C. to 94° C., and may be mixtures of copolymers derived from monomers of (i) C₃₆ diacids and (ii) ethylenediamine, and have a weight-average molecular mass of about 6000. Terminal ester groups result from esterification of the remaining acid end groups with at least one alcohol chosen from cetyl alcohol and stearyl alcohol. A mixture of cetyl and stearyl alcohols is sometimes called cetylstearyl alcohol.

Other non-limiting examples of an at least one polyamide polymer which may be used in the compositions according to the present invention include polyamide polymers resulting from the condensation of at least one aliphatic dicarboxylic acid and at least one diamine, the carbonyl and amine groups being condensed via an amide bond. In one embodiment, these polymers contain more than two carbonyl groups and more than two amine groups. Examples of these polyamide polymers are those sold under the brand name Versamid by the companies General Mills Inc. and Henkel Corp. (Versamid 930, 744 or 1655) or by the company Olin Mathieson Chemical Corp. under the brand name Onamid, in particular Onamid S or C. These resins have a weight-average molecular mass ranging from 6000 to 9000. For further information regarding these polyamides, reference may be made to U.S. Pat. Nos. 3,645,705 and 3,148,125, the disclosures of which are hereby incorporated by reference. In one embodiment, Versamid 930 or 744 may be used.

Other examples of polyamides include those sold by the company Arizona Chemical under the references Uni-Rez (2658, 2931, 2970, 2621, 2613,2624, 2665, 1554,2623 and 2662) and the product sold under the reference Macromelt 6212 by the company Henkel. For further information regarding these polyamides, reference may be made to U.S. Pat. No. 5,500,209, the disclosure of which is hereby incorporated by reference. Such polyamides display high melt viscosity characteristics. MACROMELT 6212, for example, has a high melt viscosity at 190° C. of 30-40 poise (as measured by a Brookfield Viscometer, Model RVF #3 spindle, 20 RPM).

In a further embodiment, the at least one polyamide polymer may be chosen from polyamide resins from vegetable sources. Polyamide resins from vegetable sources may be chosen from, for example, the polyamide resins of U.S. Pat. Nos. 5,783,657 and 5,998,570, the disclosures of which are herein incorporated by reference.

The at least one structuring polymer in the compositions of the invention may have a softening point greater than 50° C., such as from 65° C. to 190° C., and further such as from 70° C. to 130° C., and even further such as from 80° C. to 105° C.This softening point may be lower than that of structuring polymers used in the art which may facilitate the use of the at least one structuring polymer of the present invention and may limit the degradation of the liquid fatty phase. These polymers may be non waxy polymers.

In one embodiment, the at least one structuring polymer in the composition according to the invention corresponds to the polyamide polymers of formula (I). Due to fatty chain(s), these polymers may be readily soluble in oils and thus lead to compositions that are macroscopically homogeneous even with a high content (at least 25%) of at least one structuring polymer, unlike polymers not containing a fatty chain.

The at least one structuring polymer may be present in the composition in an amount ranging, for example, from 0.5% to 80% by weight relative to the total weight of the composition, such as for example 2% to 60%, and further, for example, from 5 to 40%. In a further embodiment the at least one structuring polymer may be present in the composition in an amount ranging, for example, from 5% to 25% by weight relative to the total weight of the composition.

When the at least one structuring polymer of the present invention comprises a urea urethane having the following formula:
R—O—CO—NH—R′—NH—CO—NH—R″—NH—CO—NH—R′—NH—CO—OR
then R represents CₙH₂ₙ₊₁, wherein n represents an integer having a value greater than 22, for example from 23 to 120, and further, for example from 23 to 68, or CₘH^{2m+1} (OCₚH₂ₚ)ᵣ—, wherein m represents an integer having a value of greater than 18, for example from 19 to 120, and further, for example, from 23 to 68, p represents an integer having a value of from 2 to 4, and r represents an integer having a value of from 1 to 10,

R′ represents: 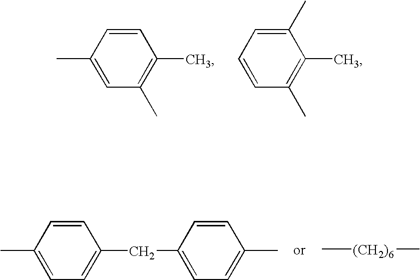
and R″ represents:

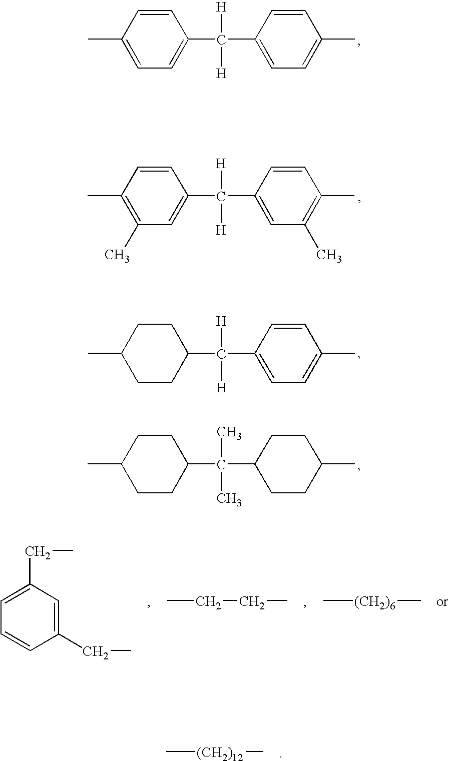

In another embodiment of the invention, the present invention is drawn to a structured composition comprising at least one liquid fatty phase structured with at least one structuring polymer comprising a polymer skeleton comprising at least one hydrocarbon-based repeating unit comprising at least one hetero atom, wherein the at least one structuring polymer further comprises at least one terminal fatty chain, optionally functionalized, chosen from alkyl and alkenyl chains, such as alkyl and alkenyl chains having at least four carbon atoms, and further such as alkyl and alkenyl chains comprising from 8 to 120 carbon atoms, bonded to the polymer skeleton via at least one linking group chosen from amines, ureas, and esters, wherein when said at least one linking group is chosen from esters, said at least one terminal fatty chain is chosen from branched alkyl groups. The at least one structuring polymer may also comprise at least one pendant fatty chain, optionally functionalized, chosen from alkyl and alkenyl chains, such as alkyl and alkenyl chains having at least four carbon atoms, and further such as alkyl and alkenyl chains comprising from 8 to 120 carbon atoms, bonded to any carbon or hetero atom of the polymer skeleton via at least one linking group chosen from amines, ureas, and esters, wherein when said at least one linking group is chosen from esters, said at least one terminal fatty chain is chosen from branched alkyl groups. The at least one structuring polymer may comprise both at least one pendant fatty chain and at least one terminal fatty chain as defined above in this paragraph.

Further, an embodiment of the invention relates to a skin, lip, or keratinous fiber care, treatment, or make-up composition comprising a structured composition containing at least one liquid fatty phase structured with at least one structuring polymer comprising a polymer skeleton comprising at least one hydrocarbon-based repeating unit comprising at least one hetero atom and at least one oil-soluble ester.

Additionally, an embodiment of the invention relates to a skin, lip, or keratinous fiber care or make-up composition comprising a structured composition containing at least one liquid fatty phase structured with at least one structuring polymer comprising a polymer skeleton comprising at least one hydrocarbon-based repeating unit comprising at least one hetero atom, at least one oil-soluble ester, and at least one coloring agent.

Additionally, an embodiment of the invention relates to a care and/or treatment and/or make-up composition for keratin materials comprising a composition containing at least one liquid fatty phase which comprises (i) at least one structuring polymer comprising a polymer skeleton which comprises at least one hydrocarbon-based repeating unit comprising at least one hetero atom; and (ii) at least one oil-soluble ester.

Another embodiment of the invention relates to a mascara, an eyeliner, a foundation, a lipstick, a blusher, a make-up-removing product, a make-up product for the body, an eyeshadow, a face powder, a concealer product, a shampoo, a conditioner, an antisun product or a care product for the lips, skin, or hair comprising a composition comprising at least one liquid fatty phase in the mascara, eyeliner, foundation, lipstick, blusher, make-up-removing product, make-up product for the body, eyeshadow, face powder, concealer product, shampoo, conditioner, antisun product or care product for the skin, lips, or hair which comprises:

(i) at least one structuring polymer comprising:

a polymer skeleton which comprises at least one hydrocarbon-based repeating unit comprising at least one hetero atom; and

(ii) at least one oil-soluble ester.

Another embodiment of the invention relates to a deodorant product or a care product for the skin or body comprising an anhydrous composition comprising at least one liquid fatty phase in the product which comprises:

(i) at least one structuring polymer comprising:

a polymer skeleton which comprises at least one hydrocarbon-based repeating unit comprising at least one hetero atom; and

(ii) at least one oil-soluble ester.

Another embodiment of the invention relates to a lipstick composition in stick form comprising at least one continuous liquid fatty phase, at least one oil-soluble ester and at least one non-waxy structuring polymer having a weight-average molecular mass of less than 100 000, the continuous liquid fatty phase, the at least one oil-soluble ester and the at least one non-waxy structuring polymer being present in the lipstick composition.

Another embodiment of the invention relates to a method for care, make-up or treatment of keratin materials comprising applying to the keratin materials an anhydrous composition comprising at least one liquid fatty phase which comprises:

(i) at least one structuring polymer comprising:

a polymer skeleton which comprises at least one hydrocarbon-based repeating unit comprising at least one hetero atom; and

(ii) at least one oil-soluble ester.

Another embodiment of the invention relates to a method for care, make-up or treatment of keratinous fibers, lips, or skin comprising applying to the keratinous fibers, lips, or skin a composition comprising at least one liquid fatty phase which comprises:

(i) at least one structuring polymer comprising:

a polymer skeleton which comprises at least one hydrocarbon-based repeating unit comprising at least one hetero atom; and

(ii) at least one oil-soluble ester.

Another embodiment of the invention relates to a method for providing an anhydrous composition having at least one property chosen from non- exudation, gloss, and comfortable deposit on keratin materials chosen from lips, skin, and keratinous fibers, comprising including in the composition at least one liquid fatty phase which comprises:

(i) at least one structuring polymer comprising:

a polymer skeleton which comprises at least one hydrocarbon-based repeating unit comprising at least one hetero atom; and

(ii) at least one oil-soluble ester.

Another embodiment of the invention relates to an anhydrous composition comprising at least one liquid fatty phase which comprises:

(i) at least one structuring polymer comprising:

a polymer skeleton which comprises at least one hydrocarbon-based repeating unit comprising at least one hetero atom; and (ii) at least one oil-soluble ester, wherein said at least one structuring polymer is not
R—O—CO—NH—R′—NH—CO—NH—R″—NH—CO—NH—R′—NH—CO—OR
wherein R represents CₙH₂ₙ₊₁—or CₘH₂ₘ₊₁ (CₚH₂ₚO)ᵣ—; n represents an integer having a value of from 4 to 22; m represents an integer having a value of from 1 to 18; p represents an integer having a value of from 2 to 4; and r represents an integer having a value of from 1 to 10;

R′ represents: 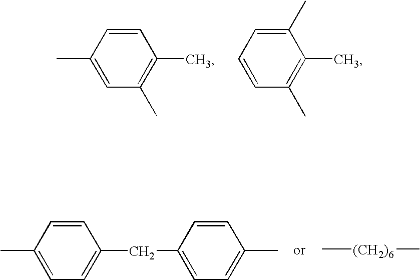
and R″ represents:

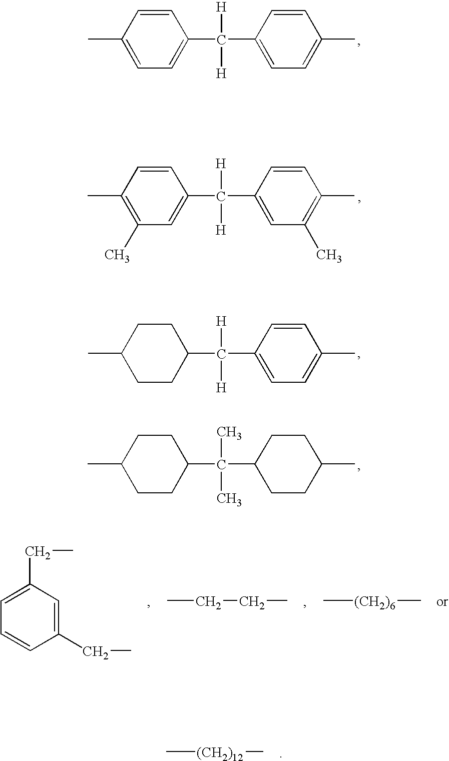

Another embodiment of the invention relates to a method of making up or caring for skin, lips keratinous fibers comprising applying to the skin, lips, or keratinous fibers a structured composition containing at least one liquid fatty phase structured with at least one structuring polymer comprising a polymer skeleton comprising at least one hydrocarbon-based repeating unit comprising at least one hetero atom and at least one oil-soluble ester.

Another embodiment of the invention relates to an anhydrous composition comprising at least one liquid fatty phase which comprises:

(i) at least one structuring polymer comprising:

a polymer skeleton which comprises at least three hydrocarbon-based repeating units comprising at least one hetero atom; and

(ii) at least one oil-soluble ester, and for example, the at least three hydrocarbon-based repeating units can be identical.

Another embodiment of the invention relates to a composition comprising at least one liquid fatty phase which comprises:

(i) at least one structuring polymer chosen from urea urethanes having the following formula:
R—O—CO—NH—R′—NH—CO—NH—R″—NH—CO—NH—R′—NH—CO—OR
wherein R represents CₙH₂ₙ₊₁—, wherein n represents an integer having a value greater than 22 or CₘH₂ₘ₊₁ (OCₚH₂ₚ)ᵣ—, wherein m represents an integer having a value of greater than 18, p represents an integer having a value of from 2 to 4, and r represents an integer having a value of from 1 to 10, R′ represents: 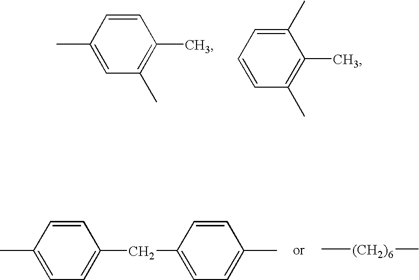
and R″ represents:

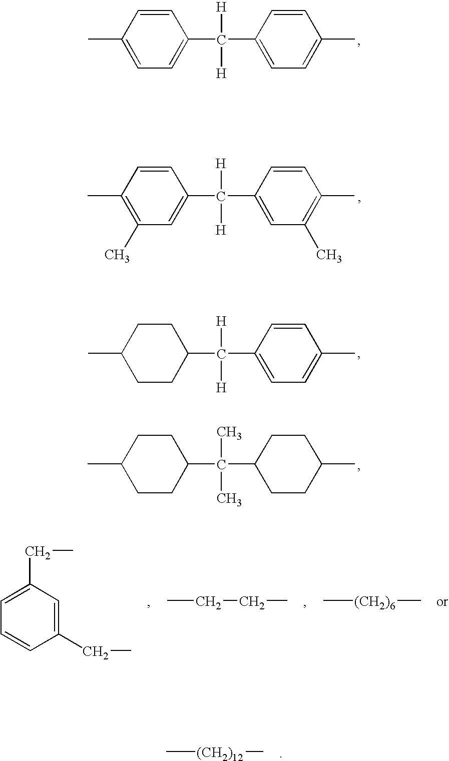

(ii) at least one oil-soluble ester.

Another embodiment of the invention relates to a composition comprising at least one liquid fatty phase which comprises:

(i) at least one structuring polymer comprising

a polymer skeleton which comprises at least one hydrocarbon-based repeating unit comprising at least one hetero atom with the proviso that the at least one hetero atom is not nitrogen; and

(ii) at least one oil-soluble ester.

The at least one structuring polymer has an affinity with the fatty phase and in particular with a chemical portion of one of the oils forming the liquid fatty phase of the composition so that physical links with the oils such as hydrogen bonds are formed.

Liquid Fatty Phase

The at least one liquid fatty phase, in one embodiment, may comprise at least one oil. The at least one oil, for example, may be chosen from polar oils and apolar oils including hydrocarbon-based liquid oils and oily liquids at room temperature. In one embodiment, the compositions of the invention comprise at least one structuring polymer and at least one polar oil. The polar oils of the invention, for example, may be added to the apolar oils, the apolar oils acting in particular as co-solvent for the polar oils.

According to the invention, the structuring of the at least one liquid fatty phase may be obtained with the aid of at least one structuring polymer, such as the polymer of formula (I). In general, the polymers of formula (I) may be in the form of mixtures of polymers, these mixtures also possibly containing a synthetic product corresponding to a compound of formula (I) in which n is 0, i.e., a diester.

The liquid fatty phase of the composition may contain more than 30%, for example, more than 40%, of liquid oil(s) having a chemical nature close to the chemical nature of the skeleton (hydrocarbon or silicone based) of the structuring polymer, and for example from 50% to 100%. In one embodiment, the liquid fatty phase structured with a polyamide-type skeleton, or polyurea, or polyurethan, or polyurea-urethane-type skeleton contains a high quantity, i.e., greater than 30%, for example greater than 40% relative to the total weight of the liquid fatty phase, or from 50% to 100%, of at least one apolar, such as hydrocarbon-based, oil. For the purposes of the invention, the expression “hydrocarbon-based oil” means an oil comprising carbon and hydrogen atoms, optionally with at least one group chosen from hydroxyl, ester, carboxyl and ether groups.

For a liquid fatty phase structured with a polymer containing a partially silicone-based skeleton, this fatty phase may contain more than 30%, for example, more than 40%, relative to the total weight of the liquid fatty phase and, for example, from 50% to 100%, of at least one silicone-based liquid oil, relative to the total weight of the liquid fatty phase.

For a liquid fatty phase structured with an apolar polymer of the hydrocarbon-based type, this fatty phase may contain more than 30%, for example more than 40% by weight, and, as a further example, from 50% to 100% by weight, of at least one liquid apolar, such as hydrocarbon-based, oil, relative to the total weight of the liquid fatty phase.

For example, the at least one polar oil useful in the invention may be chosen from:
hydrocarbon-based plant oils with a high content of triglycerides comprising fatty acid esters of glycerol in which the fatty acids may have varied chain lengths from C₄ to C₂₄, these chains possibly being chosen from linear and branched, and saturated and unsaturated chains; these oils can be chosen from, for example, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel;
synthetic oils or esters of formula R₅COOR₆ in which R₅ is chosen from linear and branched fatty acid residues containing from 1 to 40 carbon atoms and R₆ is chosen from, for example, a hydrocarbon-based chain containing from 1 to 40 carbon atoms, on condition that R₅ +R₆≧10, such as, for example, purcellin oil (cetostearyl octanoate), isononyl isononanoate, C₁₂-C₁₅ alkyl benzoates, isopropyl myristate, 2-ethylhexyl palmitate, isostearyl isostearate and alkyl or polyalkyl octanoates, decanoates or ricinoleates; hydroxylated esters such as isostearyl lactate and diisostearyl malate; and pentaerythritol esters;
synthetic ethers containing from 10 to 40 carbon atoms;
C₈ to C₂₆ fatty alcohols such as oleyl alcohol; and
C8 to C₂₆ fatty acids such as oleic acid, linolenic acid or linoleic acid.

The at least one apolar oil according to the invention is chosen from, for example, silicone oils chosen from volatile and non-volatile, linear and cyclic polydimethylsiloxanes (PDMSs) that are liquid at room temperature; polydimethylsiloxanes comprising alkyl or alkoxy groups which are pendant and/or at the end of the silicone chain, the groups each containing from 2 to 24carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof, liquid petrolatum, liquid lanolin, polydecenes, hydrogenated polyisobutene such as Parleam®), and squalane; and mixtures thereof. The structured oils, for example those structured with polyamines such as those of formula (I) or with polyurethanes, polyureas, polyurea-urethanes, in accordance with the invention, may be, in one embodiment, apolar oils, such as an oil or a mixture of hydrocarbon oils chosen from those of mineral and synthetic origin, chosen from hydrocarbons such as alkanes such as Parleam® oil, isoparaffins including isododecane, and squalane, and mixtures thereof. These oils may, in one embodiment, be combined with at least one phenylsilicone oil.

The liquid fatty phase, in one embodiment, contains at least one non-volatile oil chosen from, for example, hydrocarbon-based oils of mineral, plant and synthetic origin, synthetic esters or ethers, silicone oils and mixtures thereof.

In practice, the total liquid fatty phase can be, for example, present in an amount ranging from 1% to 99% by weight relative to the total weight of the composition, for example from 5% to 95.5%, from 10% to 80%, or from 20% to 75%.

For the purposes of the invention, the expression “volatile solvent or oil” means any non-aqueous medium capable of evaporating on contact with the skin or the lips in less than one hour at room temperature and atmospheric pressure. The volatile solvent(s) of the invention is(are) organic solvents, such as volatile cosmetic oils that are liquid at room temperature, having a non-zero vapor pressure, at room temperature and atmospheric pressure, ranging in particular from 10⁻² to 300 mmHg and, for example, greater than 0.3 mmHg. The expression “non-volatile oil” means an oil which remains on the skin or the lips at room temperature and atmospheric pressure for at least several hours, such as those having a vapor pressure of less than 10⁻² mmHg.

According to the invention, these volatile solvents may facilitate the staying power or long wearing properties of the composition on the skin, the lips or superficial body growths, such as keratinous fibers. The solvents can be chosen from hydrocarbon-based solvents, silicone solvents optionally comprising alkyl or alkoxy groups that are pendant or at the end of a silicone chain, and a mixture of these solvents.

The volatile oil(s), in one embodiment, is present in an amount ranging up to 95.5% relative to the total weight of the composition, such as from 2% to 75%, and, as a further example, from 10% to 45%. This amount will be adapted by a person skilled in the art according to the desired staying power or long wearing properties.

The at least one liquid fatty phase of the compositions of the invention may further comprise a dispersion of lipid vesicles. The compositions of the invention may also, for example, be in the form of a fluid anhydrous gel, a rigid anhydrous gel, a fluid simple emulsion, a fluid multiple emulsion, a rigid simple emulsion or a rigid multiple emulsion. The simple emulsion or multiple emulsion may comprise a continuous phase chosen from an aqueous phase optionally containing dispersed lipid vesicles or oil droplets, or a fatty phase optionally containing dispersed lipid vesicles or water droplets. In one embodiment, the composition has a continuous oily phase or fatty phase and is more specifically an anhydrous composition, for example, a stick or dish form. An anhydrous composition is one that has less than 10% water by weight, such as, for example, less than 5% by weight.

Oil-Soluble Ester

The compositions of the invention may also comprise at least one oil-soluble ester comprising at least one free hydroxy group. Any oil-soluble ester comprising at least one free hydroxy group may be within the practice of the invention with the proviso that said at least one oil-soluble ester is not castor oil. While a composition of the invention may, for example, further comprise castor oil, castor oil alone in combination with at least one structuring polymer is not within the practice of the invention. Also, in one embodiment, the at least one oil-soluble ester is not sucrose acetate isobutyrate.

The at least one oil-soluble ester comprising at least one free hydroxy group may be chosen from, for example, propylene glycol ricinoleate, isopropyl hydroxystearate, triisocetyl citrate, diisostearyl malate, octyl hydroxystearate, triisoarachidyl citrate, cetyl lactate, dioctyl malate, octyldodecyl hydroxystearate, di-isostearyl malate, and di-isostearyl lactate.

In one embodiment, the compositions of the invention may also comprise at least one oil-soluble ester comprising at least one free hydroxy group with the proviso that said at least one oil-soluble ester is not sucrose acetate isobutyrate.

In one embodiment, the at least one oil-soluble ester comprising at least one free hydroxy group, such as diisostearyl malate and triisocetyl citrate may add stability. For example, the use of these esters may minimize oil droplet formulation at room temperature and elevated temperature storage. The introduction of at least one hydroxy bearing ester, in addition, may dramatically improve the overall softening point of the finished clear anhydrous stick.

In a further embodiment, certain at least one oil-soluble esters comprising at least one free hydroxy group may provide the firmest and clearest compositions and sticks and may also improve the gelling efficiency in relation to a composition comprising structuring polymers alone. For example, a composition comprising 16-20% structuring polymer with the at least one oil-soluble ester comprising at least one free hydroxy group chosen from isopropyl hydroxystearate had excellent clarity and structure.

Depending on the at least one structuring polymer and its concentration and the at least one oil-soluble ester comprising at least one free hydroxy group and its concentration, some compositions may develop syneresis after aging for one day at 25° C., which may be disadvantageous in certain embodiments. The skilled artisan may be able to cure this defect by varying the at least one structuring polymer and/or the at least one oil-soluble ester comprising at least one free hydroxy group. The skilled artisan may also be able to cure this defect by varying the concentration of at least one of these ingredients.

In one embodiment, the at least one oil-soluble ester comprising at least one free hydroxy group may be present in the composition in an amount ranging from 10% to 84% by weight relative to the total weight of the composition, such as, for example 20% to 70%.

The concentrations of the at least one structuring polymer and of the at least one oil-soluble ester, however, may be chosen according to the desired hardness and desired stability of the compositions and according to the specific application envisaged. The respective concentrations of the at least one structuring polymer and of the at least one oil-soluble ester can be such that a disintegrable solid which does not flow under its own weight is obtained.

Depending on the intended application, such as a stick, hardness of the composition may also be considered. The hardness of a composition may, for example, be expressed in grams (g). The composition of the present invention may, for example, have a hardness ranging from 20 g to 2000 g, such as from 20 g to 900 g, and further such as from 20 g to 600 g. This hardness is measured in one of two ways. A first test for hardness is according to a method of penetrating a probe into said composition and in particular using a texture analyzer (for example TA-XT2 from Rhéo) equipped with an ebonite cylinder of height 25 mm and diameter 8 mm. The hardness measurement is carried out at 20° C. at the center of 5 samples of said composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 g

The second test for hardness is the “cheese wire” method, which involves cutting an 8.1 mm tube of composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 g to 300 g, such as from 30 g to 250 g, and further such as from 30 g to 200 g.

The hardness of the composition of the present invention may be such that the compositions are self-supporting and can easily disintegrate to form a satisfactory deposit on a keratinous material. In addition, this hardness may impart good impact strength to the inventive compositions which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within the scope of the invention.

According to the present invention, the compositions in stick form may also possess the properties of deformable, flexible elastic solids and may also have noteworthy elastic softness upon application to a keratinous material. The compositions in stick form of the prior art do not have this elasticity and flexibility.

Fatty Alcohol

The compositions of the invention may further comprise at least one fatty alcohol. The at least one fatty alcohol may be chosen from, for example, C₈ to C₂₆, such as from, C₂ to C₂₂ fatty alcohols. In one embodiment, the at least one fatty alcohol is chosen from myristyl, cetyl, stearyl, and behenyl alcohol. The fatty alcohols may, for example, be present in the composition in an amount ranging from 0.1% to 15.0% by weight, relative to the total weight of the composition, such as, for example, 0.5% to 10% and 05% to 8.0%. In a further embodiment, the skilled artisan may be able to cure a stability defect by the addition of at least one fatty alcohol to the composition. For example, the addition of at least one fatty alcohol may improve stick structure, minimize syneresis, and generally improve application properties without interfering with stick transparency, as compared to a composition that does not contain the at least one fatty alcohol.

Oil-Soluble Polymer

The compositions of the invention may further comprise at least one oil-soluble polymer chosen from alkylated guar gums and alkyl celluloses. Alkylated guar gums include, for example, ethyl guars and C₁₋₅ alkyl galactomannans, such as N-HANCE AG-50 and N-HANCE AG-200 from Aqualon. An alkyl cellulose, may be chosen from, for example, ethylcellulose (such as ETHOCEL, from Dow Chemical). In one embodiment, the at least one oil-soluble polymer may be present in the composition in an amount ranging from 0.05% to 10.0% by weight relative to the total weight of the composition, such as, for example 0.1% to 5% and 0.1% to 3%. These ingredients can further stabilize, for example, a clear sunscreen complex composition against syneresis.

In one embodiment, a composition according to the invention may be stabilized by the inclusion of at least one oil-soluble polymer chosen from alkyl celluloses. In a further embodiment, at least one alky galactomannan, such as N-HANCE AG-50, may be used to stabilize a stick composition against stick syneresis, particularly at elevated temperatures such as, for example, 45° C.

Oil-Soluble Cationic Surfactant

As described above, the compositions of the invention further comprise at least one oil-soluble cationic surfactant. In one embodiment, the at least one oil-soluble cationic surfactant may be chosen from lauryl methyl gluceth-10-hydroxypropyl dimmonium chloride, which may impart cosmetic elegance to a composition. The at least one oil-soluble cationic surfactant may also, for example, be chosen from quaternary ammonium compounds including salts of quaternary ammonium compounds and fatty amines including salts of fatty amines.

In one embodiment, the at least one oil-soluble cationic surfactant is chosen from water-insoluble surfactants of the formula 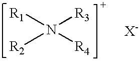
wherein R₁ -R₄ is independently chosen from an aliphatic group of from 1 to 22 carbon atoms, C₁-C₃ alkyl, hydroxyalkyl, polyalkoxy, aromatic, aryl, and alkylaryl groups having from 12 to 22 carbon atoms; X is chosen from halogen, acetate, phosphate, nitrate, and alkylsulfate radicals. The aliphatic groups may, for example, contain in addition to carbon and hydrogen atoms, ether linkages, and other groups such as amino groups.

The at least one oil-soluble cationic surfactant may also, for example, be chosen from quatemary ammonium salts of the formula 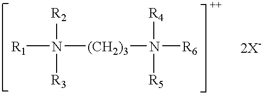
wherein R₁ is an aliphatic group having from 16 to 22 carbon atoms; R₂, R₃, R₄, R₅, and R₆ are independently chosen from hydrogen and alkyl having from 1 to 4 carbon atoms, and X is chosen from halogen, acetate, phosphate, nitrate, and alkyl sulfate radicals. The at least one oil-soluble cationic surfactant may, for example, be tallow propane diammonium dichloride.

Non-limiting examples of the at least one oil-soluble cationic surfactant include the quatemary ammonium salts: dialkyldimethyl-ammonium chlorides, wherein the alkyl groups have from 12 to 22 carbon atoms and are derived from long-chain fatty acids, such as hydrogenated tallow fatty acid (tallow fatty acids yield quaternary compounds wherein R₁ and R₂ have predominately from 16 to 18 carbon atoms); ditallowdimethyl ammonium chloride; ditallowdimethyl ammonium methyl sulfate; dihexadecyl dimethyl ammonium chloride; di(hydrogenated tallow) dimethyl ammonium chloride; dioctadecyl dimethyl ammonium chloride; dieicosyl dimethyl ammonium chloride; didocosyl dimethyl ammonium chloride; di(hydrogenated tallow) dimethyl ammonium acetate; dihexadecyl dimethyl ammonium chloride, dihexadecyl dimethyl ammonium acetate; ditallow dipropyl ammonium phosphate; ditallow dimethyl ammonium nitrate; di(coconutalkyl) dimethyl ammonium chloride; diceltyl dimethyl ammonium chloride; stearyl dimethyl benzyl ammonium chloride; behenyl trimethyl ammonium chloride; Di-(hydrogenated tallow) dimethyl ammonium chloride.

Non-limiting examples of the at least one oil-soluble cationic surfactant also include salts of primary, secondary, and tertiary fatty amines. In one embodiment,salts of amines may comprise alkyl groups having from 12 to 22 carbon atoms, and may be substituted and unsubstituted. Amines may be chosen from, for example, stearamido propyl dimethyl amine, diethyl amino ethyl stearamide, dimethyl stearamine, dimethyl soyamine, soyamine, tridecyl amine, ethyl stearylamine, ethoxylated (2 moles E.O.) stearylamine, dihydroxyethyl stearylamine, and arachidylbehenylamine. Amine salts may be chosen from, for example, halogens, actetates, phosphates, nitrates, citrates, lactates, and alkyl sulfates. In one embodiment, the amine salts are chosen from stearylamine hydrochloride, soyamine chloride, stearylamine formate, N-tallowpropane diaminedichloride, and stearamidopropyl dimethylamine citrate. The at least one oil-soluble cationic surfactant may also be chosen from cationic amine surfactants disclosed in U.S. Pat. No. 4,275,055, the disclosure of which is hereby incorporated by reference.

In another embodiment, the at least one oil-soluble cationic surfactant may be chosen from quatemary imidazolinium compounds including quaternary imidazolinium salts. Quatemary imidazolinium compounds include, for example, imidazolinium compounds containing C₁₂-C₂₂ alkyl groups such as
1-methyl-1-[(stearoylamide)ethyl]-2-heptadecyl4,5-dihydroimidazolinium chloride,
1 -methyl-1-[(palmitoylamide)ethyl]-2-octadecyl4,5-dihydroimidazolinium chloride and
1-methyl-1-[(tallowamide)-ethyl]-2-tallow-imidazolinium methyl sulfate. The at least one oil-soluble cationic surfactant may also be chosen from conditioning agents that are disclosed in U.S. Pat. No. 4,387,090, to Bolich, issued Jun. 7, 1983, which is incorporated by reference herein.

The at least one oil-soluble cationic surfactant may be present in the composition, for example, in an amount ranging from 0.1% to 10% by weight relative to the weight of the composition, such as, for example, 0.1% to 5.0% and 0.5% to 2.0%. As used herein cosmetic elegance refers to substantially low tackiness, ease of application, or elegant feel.

Wax

According to another embodiment, the compositions of the invention may further comprise at least one wax. At least one wax, for example, may be used to form a non-transparent composition. As used herein, a “wax” may be any lipophilic fatty compound which is soluble in the liquid fatty phase, unlike most fillers or pigments. The at least one wax, for example, may have a melting point greater than about 45°, such as, for example greater than about 55° C. Non-limiting examples of such waxes include waxes of natural origin, such as beeswax, carnauba wax, candelilla wax, ouricury wax, Japan wax, cork fiber wax, sugar cane wax, paraffin waxes, lignite wax, microcrystalline waxes, lanolin wax, montan wax and ozokerites, hydrogenated oils such as hydrogenated jojoba oil, jojoba esters, waxes of synthetic origin, such as polyethylene waxes derived from polymerization of ethylene, waxes obtained by Fischer-Tropsch synthesis, fatty acid esters and glycerides, and silicone waxes such as derivatives of poly(di)methylsiloxane. In one embodiment, the at least one wax may be present in the composition up to 3%, and in another embodiment at least 3%, such as up to 30% or up to 50%.

As described above, the composition may also comprise at least one liquid fatty phase wherein the liquid fatty phase comprises at least one structuring polymer and at least one oil-soluble ester comprising at least one free hydroxy group with the proviso that said at least one oil-soluble ester is not castor oil. In one embodiment, the liquid fatty phase may further comprise additional ingredients chosen from at least one fatty alcohol.

The composition of the present invention, in one embodiment, may comprise a physiologically acceptable medium. The composition may also further comprise at least one suitable additive commonly used in the field concerned chosen from coloring agents, antioxidants, essential oils, preserving agents, fragrances, neutralizing agents, liposoluble polymers, and cosmetically active agents and dermatological active agents such as, for example, emollients, moisturizers, vitamins, essential fatty acids and sunscreens. The at least one additive is generally present in a concentration ranging from 0% to 20% by weight of the total weight of the composition, such as from 0% to 10%.

Needless to say, the person skilled in the art will take care to select the optional additional additives and the amount thereof such that at least one advantageous property of the composition according to the invention, such as stability, is not, or is not substantially, adversely affected by the addition(s) envisaged.

The compositions of the invention may also comprise at least one coloring agent chosen from pigments, dyes, nacres, and pearling agents. The at least one coloring agent may be chosen, for example, in order to obtain make-up compositions which give good coverage, that is, which do not leave a significant amount of the at least one keratin material to which it is applied showing through. The pigments may also reduce the sticky feel of the compositions, unlike soluble dyes.

Representative liposoluble dyes which may be used according to the present invention include Sudan red, DC Red 17, DC Green 64, β-carotene, soybean oil, Sudan brown, DC Yellow 11, DC Violet 2, DC Orange 5, quinoline yellow, and annatto. The liposoluble dyes, when present, generally have a concentration ranging up to 20% by weight of the total weight of the composition, such as from 0.1% to 6%.

The pigments which may be used according to the present invention may be chosen from white, colored, mineral, organic, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate and ferric blue. Representative examples of organic pigments include carbon black, pigments of D & C type, and lakes based on cochineal carmine, barium, strontium, calcium and aluminum. If present, the pigments may have a concentration ranging up to 40% by weight of the total weight of the composition, such as from 1% to 35%, and further such as from 2% to 25%.

The nacreous pigments (or nacres) which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacres, if present, may have a concentration ranging up to 30% by weight of the total weight of the composition, such as from 0.1% to 20%.

Another invention disclosed herein provides a composition comprising at least one structuring polymer, as described herein, and at least one UV blocker. The composition may further comprise a physiologically acceptable medium. The at least one UV blocker, may be chosen from, for example, organic filters including lipophilic organic filters, inorganic nanoparticles and mixtures thereof. The UV blocker, in one embodiment, is present in an amount ranging from 0.1% to 30% of the total weight of the composition, such as, for example 0.5% to 15%.

The packaging and application device for any subject of the invention may be chosen and manufactured by persons skilled in the art on the basis of their general knowledge, and adapted according to the nature of the composition to be packaged. Indeed, the type of device to be used may be in particular linked to the consistency of the composition, in particular to its viscosity; it may also depend on the nature of the constituents present in the composition, such as the presence of volatile compounds.

The invention will be illustrated by, but is not intended to be limited to, the following examples.

### EXAMPLE 1

### Clear Anhydrous Sunscreen Stick

TABLE I

| RAW MATERIALS | Phase | A | B | C | D | E |
|---|---|---|---|---|---|---|
| Schercemol DISM | A | 10 | 10 | 10 | 10 | 10 |
| (Diisostearyl | | | | | | |
| malate) | | | | | | |
| Ceraphyl 45 | A | 10 | 10 | 20 | 20 | 20 |
| (Dioctyl malate) | | | | | | |
| Cristal 0 | A | 33 | 32.95 | 30.6 | 29.9 | 29 |
| (Castor oil) | | | | | | |
| NatureChem PGR | A | 10.5 | 10.5 | 10.5 | 10.5 | 10.5 |
| (Propylene glycol | | | | | | |
| ricinoleate) | | | | | | |
| Macromelt 6212 | B | 16 | 16 | 16 | 16 | 16 |
| (Polyamide resin) | | | | | | |
| Cetyl Alcohol | C | — | — | 2 | 3 | 4 |
| Others*₁ | E | — | 0.05 | 0.4 | 0.1 | — |
| Uvinul M40 USP | D | 3 | 3 | 3 | 3 | 3 |
| (Benzophenone-3) | | | | | | |
| Parsol MCX | D | 7.5 | 7.5 | 7.5 | 7.5 | 7.5 |
| (Octyl methoxy | | | | | | |
| cinnamate) | | | | | | |

*₁Others: Preservatives, masking agents, colorants, vitamins, oil-soluble actives, anti-oxidants, and dermatological actives.

The compositions of table 1 were prepared using the following procedure. The ingredients of phase A were added to a main vessel and heated to 110-115° C. while mixing with the aid of an impeller mixer. At 110-115° C. phase B was added to phase A with continued mixing. The beads of polyamide resin were allowed to dissolve and the mixture was removed from the heat and cooled to 80-82° C. Phases C, D, and E were added to the AB mixture while maintaining the temperature at about 80-82° C. with slow impeller mixing. The compositions were mixed until homogeneous (about 1 minute), then used to fill a suitable container or mold.

The resulting compositions were firm at room temperature (25° C.). A very fine uniform oil coat covered the surface of some of the compositions, however, none of the compositions failed the stability test. At elevated temperatures (45° C.), the overall structure and stick characteristics remained unchanged. There was a moderate oil coat on the surface of the stick structure of some of the compositions, however, none of the compositions failed the stability test.

### EXAMPLE 2

### Clear Anhydrous Sunscreen Stick with an Oil-Soluble Cationic Polymer

TABLE 2

| RAW MATERIALS | Phase | A | B | C |
|---|---|---|---|---|
| Schercemol DISM | A | 10 | 10 | 10 |
| (Diisostearyl malate) | | | | |
| Ceraphyl 45 | A | 20 | 200 | 20 |
| (Dioctyl malate) | | | | |
| Cristal 0 | A | 28.9 | 27.9 | 29.4 |
| (Castor Oil) | | | | |
| NatureChem PGR | A | 10.5 | 10.5 | 10.5 |
| (Propylene glycol ricinoleate) | | | | |
| Glucquat - 100 | A | 1 | 2 | 0.5 |
| (Lauryl methyl gluceth-10 | | | | |
| hydroxypropyl dimonium chloride) | | | | |
| Macromelt 6212 | B | 16 | 16 | 16 |
| (Polyamide resin) | | | | |
| Cetyl Alcohol | C | 3 | 3 | 3 |
| Propyl Paraben | C | 0.1 | 0.1 | 0.1 |
| Uvinul M40 USP | D | 3 | 3 | 3 |
| (Benzophenone-3) | | | | |
| Parsol MCX | D | 7.5 | 7.5 | 7.5 |
| (Octyl methoxy cinnamate) | | | | |

The compositions of table 2 were prepared using the following procedure. The ingredients of phase A were added to a main vessel and heated to 110-115° C. while mixing with the aid of an impeller mixer. At 110-115° C. phase B was added to phase A with continued mixing. The beads of polyamide resin were allowed to dissolve and the mixture was removed from the heat and cooled to 80-82° C.Phases C, and D were added to the AB mixture while maintaining the temperature at about 80-82° C. with slow impeller mixing. The compositions were mixed until homogeneous (about 1 minute), then used to fill a suitable container or mold.

The resulting compositions were firm at room temperature (25° C.). A very fine uniform oil coat covered the surface of some of the compositions, however, none of the compositions failed the stability test. At elevated temperatures (45° C.), the overall structure and stick characteristics remained unchanged. There was a moderate oil coat on the surface of the stick structure of some of the compositions, however, none of the compositions failed the stability test.

### EXAMPLE 3

### Clear Anhydrous Sunscreen Sticks with an Oil-Soluble Cationic Polymer

TABLE 3

| RAW MATERIALS | Phase | A | B | C | D | E |
|---|---|---|---|---|---|---|
| Schercemol DISM | A | 10 | 10 | 10 | 10 | 10 |
| (Diisostearyl malate) | | | | | | |
| Ceraphyl 45 | A | 20 | 20 | 20 | 20 | 20 |
| (Dioctyl malate) | | | | | | |
| Cristal 0 | A | 26.15 | 24.15 | 22.9 | 23.9 | 23.15 |
| (Caster Oil) | | | | | | |
| NatureChem PGR | A | 10.5 | 10.5 | 10.5 | 10.5 | 10.5 |
| (Propylene glycol ricinoleate) | | | | | | |
| Macromelt 6212 | B | 16 | 16 | 16 | 16 | 16 |
| (Polyamide resin) | | | | | | |
| N-Hance-AG-50 | A | — | 2 | — | — | — |
| (C₁-C₅ alkyl galactomannan) | | | | | | |
| N-Hance-AG-200 | A | — | — | 3 | — | — |
| (C₁-C₅ alkyl galactomannan) | | | | | | |
| Ethocel 100 | A | — | — | — | 2 | — |
| (Ethyl cellulose) | | | | | | |
| Ethocel 7 | A | — | — | — | — | 3 |
| (Ethyl cellulose) | | | | | | |
| Cetyl Alcohol | C | 4 | 4 | 4 | 4 | 4 |
| Propyl Paraben | C | 0.1 | 01 | 0.1 | 0.1 | 0.1 |
| Parsol 1789 | D | 3 | 3 | 3 | 3 | 3 |
| (Butyl methoxydibenzol | | | | | | |
| methane) | | | | | | |
| Neo Heliopan 303 | D | 10 | 10 | 10 | 10 | 10 |
| (Octocrylene) | | | | | | |
| Flavoring Oil | E | 0.25 | 0.25 | 0.5 | 0.5 | 0.25 |

The compositions of table 3 were prepared using the following procedure. The ingredients of phase A were added to a main vessel and heated to 110-115° C. while mixing with the aid of an impeller mixer. At 110-115° C. phase B was added to phase A with continued mixing. The beads of polyamide resin were allowed to dissolve and the mixture was removed from the heat and cooled to 80-82° C.Phases C, D, and E were added to the AB mixture while maintaining the temperature at about 80-82° C. with slow impeller mixing. The compositions were mixed until homogeneous (about 1 minute), then used to fill a suitsable container or mold.

The resulting compositions were firm at room temperature (25° ). A very fine uniform oil coat covered the surface of some of the compositions, however, none of the compositions failed the stability test. At elevated temperature (45° C.), the overall structure and stick characteristics remained unchanged. There was a moderate oilcoated on the surface of the stick structure, however, none of the compositions failed the stability test.

### EXAMPLE 4

### Lipsticks with an Oil-Soluble Cationic Polymer

TABLE 4

| RAW MATERIALS | A | B |
|---|---|---|
| Schercemol DISM | q.s.p | 12 |
| (Diisostearyl malate) | | |
| Octyldocecanol | 10 | — |
| Isononyl Isononanoate | 5.9 | qsp |
| Polyglycerol-2 Diisostearate | 5.9 | 5.9 |
| Uniclear (Ethylenediamine/Tall | 15 | 15 |
| Oil Dimer Acid/Stearyl | | |
| Alcohol Copolymer) | | |
| Polyethylene | 3 | 12 |
| Iron Oxides | 4 | 4 |
| Nylon-12 | 3 | 4 |
| Red 7 Lake | 1.8 | 1.8 |
| Titanium Dioxide | 1.2 | 1.2 |
| Barium sulfate | 0.6 | 0.6 |
| Rosin/Colophonium Tetradibutyl | 0.6 | 0.6 |
| Pentaerythrityl | | |
| Hydroxyhydrocinnamate | 0.05 | 0.05 |

The compositions were mixed until homogeneous, then used to fill a suitable container or mold.

### EXAMPLE 5

### Anhydrous Gel Lip Compositions

TABLE 5

| RAW MATERIALS | | A | B | C |
|---|---|---|---|---|
| Ethylenediamine/Tall Oil Dimer Acid/Stearyl | | 25 | 25 | 25 |
| Alcohol Copolymer | | | | |
| Hydrogenated Polyisobutene | | 62 | 58 | 55.5 |
| Octyldodecanol | | 10 | 10 | 10 |
| Hydrogenated polyisobutene | 45% | 0.01 | 0.01 | 0.01 |
| Polyhydroxystearic acid | 5% | | | |
| Yellow 5 lake | 50% | | | |

| | | | |
|---|---|---|---|
| Ethylhexyl methoxycinnamate | 1 | 5 | 7.5 |
| Fragence | 2 | 2 | 2 |

The compositions were mixed until homogeneous, then used to fill a suitable container or mold.

1-299. (Canceled).

300. A method for providing stability to a cosmetic composition comprising including in said cosmetic composition at least one liquid fatty phase which comprises:
(i) at least one structuring polymer chosen from polyamide polymers of formula (I): 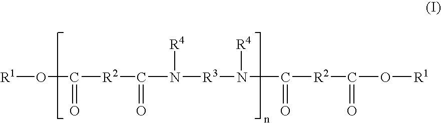
in which:
n is an integer which represents the number of amide units such that the number of ester groups present in said at least one structuring polymer ranges from 10% to 50% of the total number of all said ester groups and all said amide groups comprised in said at least one structuring polymer;
R¹, which are identical or different, are each chosen from alkyl groups having at least 4 carbon atoms and alkenyl groups having at least 4 carbon atoms;
R², which are identical or different, are each chosen from C₄ to C₄₂ hydrocarbon-based groups with the proviso that at least 50% of R² are chosen from C₃₀ to C₄₂ hydrocarbon-based groups;
R³, which are identical or different, are each chosen from C₂ to C₃₆ hydrocarbon-based groups; and
R⁴, which are identical or different, are each chosen from hydrogen and C₁ to C₁₀ alkyl groups, with the proviso that at least 50% of all R⁴ are chosen from hydrogen;
(ii) at least one oil-soluble ester comprising at least one free hydroxy group with the proviso that said at least one oil-soluble ester is not castor oil; and
(iii) at least one coloring agent.

301. The method according to claim 300, wherein the at least one structuring polymer is chosen from ethylenediamine/stearyl dimer tallate copolymer.

302. The method according to claim 300, wherein said composition further comprises at least one additional fatty material.

303. The method according to claim 302, wherein said at least one additional fatty material is chosen from gums, fatty materials pasty at ambient temperature, and resins.

304. The method according to claim 300, wherein said composition further comprises at least one fatty alcohol.

305. The method according to claim 304, wherein said at least one fatty alcohol is chosen from C₈ to C₂₆ fatty alcohols.

306. The method according to claim 305, wherein said at least one fatty alcohol is chosen from C₁₂ to C₂₀ fatty alcohols.

307. The method according to claim 306, wherein said C₁₂ to C₂₀ fatty alcohols are chosen from myristyl alcohol, cetyl alcohol, stearyl alcohol and behenyl alcohol.

308. The method according to claim 304, wherein the at least one fatty alcohol is present in a concentration ranging from 0.1% to 15.0% by weight, relative to the weight of the composition.

309. The method according to claim 308, wherein the at least one fatty alcohol is present in a concentration ranging from 0.5% to 10.0% by weight, relative to the weight of the composition.

310. The method according to claim 309 wherein the at least one fatty alcohol is present in a concentration ranging from 0.5% to 8.0% by weight, relative to the weight of the composition.

311. The method according to claim 300, wherein said composition further comprises at least one oil-soluble polymer.

312. The method according to claim 311, wherein said at least one oil-soluble polymer is chosen from alkylated guar gums and alkyl celluloses.

313. The method according to claim 311, wherein the at least one oil-soluble polymer is present in a concentration ranging from 0.05% to 10% by weight, relative to the weight of the composition.

314. The method according to claim 313, wherein the at least one oil-soluble polymer is present in a concentration ranging from 0.1% to 5% by weight, relative to the weight of the composition.

315. The method according to claim 314 wherein the at least one oil-soluble polymer is present in a concentration ranging from 0.1% to 3% by weight, relative to the weight of the composition.

316. The method according to claim 300, wherein said composition further comprises at least one wax.

317. The method according to claim 316, wherein said at least one wax is chosen from carnauba wax, candelilla wax, ouricury wax, Japan wax, cork fiber wax, sugar cane wax, paraffin waxes, lignite wax, microcrystalline waxes, lanolin wax, montan wax, polyethylene waxes, waxes obtained by Fischer-Tropsch synthesis, silicone waxes, ozokerites, hydrogenated jojoba oil, fatty acid esters, and fatty acid ester glycerides.

318. The method according to claim 316, wherein said at least one wax is present at a concentration of up to 3% relative to the total weight of said composition.

319. The method according to claim 300, wherein the composition further comprises at least one preserving agent chosen from methylparaben, ethylparaben, propylparaben, and butylparaben.

320. The method according to claim 300, wherein the at least one structuring polymer is chosen from ethylenediamine/stearyl dimer dilinoleate copolymer.

321. A container comprising a lipstick composition comprising:
(i) at least one structuring polymer chosen from polyamide polymers of formula (I): 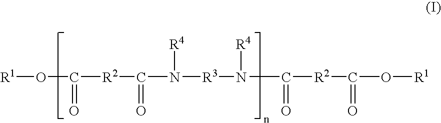
in which:
n is an integer which represents the number of amide units such that the number of ester groups present in said at least one structuring polymer ranges from 10% to 50% of the total number of all said ester groups and all said amide groups comprised in said at least one structuring polymer;
R¹, which are identical or different, are each chosen from alkyl groups having at least 4 carbon atoms and alkenyl groups having at least 4 carbon atoms;
R², which are identical or different, are each chosen from C₄ to C₄₂ hydrocarbon-based groups with the proviso that at least 50% of R² are chosen from C₃₀ to C₄₂ hydrocarbon-based groups;
R³, which are identical or different, are each chosen from C2 to C₃₆ hydrocarbon-based groups; and
R⁴, which are identical or different, are each chosen from hydrogen and C₁ to C₁₀ alkyl groups, with the proviso that at least 50% of all R⁴ are chosen from hydrogen;
(ii) at least one oil-soluble ester comprising at least one free hydroxy group with the proviso that said at least one oil-soluble ester is not castor oil; and
(iii) at least one coloring agent.
